(19) United States
(12) Patent Application Publication
(10) Pub. No.: US 2025/0336001 A1
(43) Pub. Date: Oct. 30, 2025

(54) IDENTIFYING INCONSISTENCIES IN DATA FOR UNDERWRITING

(71) Applicant: Sixfold AI, Inc., New York, NY (US)

(72) Inventors: Alexander M. Schmelkin, New York, NY (US); Brian Moseley, New York, NY (US); Jane Tran, New York, NY (US); Ian Namit Hirschfeld, New York, NY (US); Gregory Hess Tourville, New York, NY (US); Dhrubajyoti Das, New York, NY (US); Stewart Hu, New York, NY (US)

(21) Appl. No.: 18/651,234

(22) Filed: Apr. 30, 2024

(51) Int. Cl. G06Q 40/08; G16H 10/60

(52) U.S. Cl. CPC G06Q 40/08; G16H 10/60

(57) ABSTRACT

Systems herein describe an inconsistency detection system to improve insurance underwriting. The inconsistency detection system accesses medical records associated with a patient user, extracts data from the medical records, compares the extracted data to a database of medical data, identifies inconsistencies between the extracted data and the database of medical records, based on the identified inconsistencies, automatically generates a textual summary of the identified inconsistencies and displays the identified inconsistencies, and the generated textual summary of the identified inconsistencies to a user.

The present disclosure generally relates to insurance underwriting. More specifically, but not by way of limitations, embodiments herein describe identifying inconsistencies in data for underwriting.

### BACKGROUND

Insurance underwriting involves evaluating a risk to determine if an insurer will insure an insured party. The process requires an analysis of various factors to determine a cost associated with paying out an insurance claim.

### BRIEF DESCRIPTION OF THE SEVERAL VIEWS OF THE DRAWINGS

In the drawings, which are not necessarily drawn to scale, like numerals may describe similar components in different views. To easily identify the discussion of any particular element or act, the most significant digit or digits in a reference number refer to the figure number in which that element is first introduced. Some non-limiting examples are illustrated in the figures of the accompanying drawings in which:

FIG. 1 is a diagrammatic representation of an insurance analytics system 100 in a networked environment in which the present disclosure may be deployed, in accordance with some examples.

FIG. 2 is a diagrammatic representation of an insurance analytics system, in accordance with some examples, that has both client-side and server-side functionality.

FIG. 3 is a diagrammatic representation of a data structure as maintained in a database, in accordance with some examples.

FIG. 4 is a diagrammatic representation of the details of the inconsistency detection system in accordance with one embodiment.

FIG. 5 illustrates a process for identifying inconsistencies in an insurance application, in accordance with some examples.

FIGS. 6-8 illustrates the inconsistency detection system interface in accordance with some examples.

FIG. 9 is a diagrammatic representation of a machine in the form of a computer system within which a set of instructions may be executed for causing the machine to perform any one or more of the methodologies discussed herein, in accordance with some examples.

FIG. 10 is a block diagram showing a software architecture within which examples may be implemented.

### DETAILED DESCRIPTION

When an insurance company or financial institution determine whether to provide a loan, insurance policy or investment to an applicant, a human underwriter employed by the insurance company or financial institution has to assess the risk associated with the financial venture and the applicant and determine whether the risk is within parameters set by his or her employer.

Occasionally, there are inconsistencies in the data which can impact the risk associated with the insured party. The paragraphs below describe an inconsistency detection system for detecting and identifying inconsistencies across medical records during the underwriting process. The inconsistency detection system uses natural language processing and machine learning techniques to process the medical records, extract data and analyzed the extracted data to identify inconsistencies across the data sources. By automatically identifying and flagging the inconsistencies across the various data sources, the inconsistency detection system improves accuracy during the insurance underwriting process and provides succinct explanations to underwriters as they make policy decisions based on automated recommendations generated by the inconsistency detection system.

Thus, by providing the reasoning behind the recommendations and results generated by the system to the customer or underwriter, the inconsistency detection system thus enhances capacity, accuracy and transparency of the underwriting process. Further details of the inconsistency detection system are described below.

### NETWORKED COMPUTING ENVIRONMENT

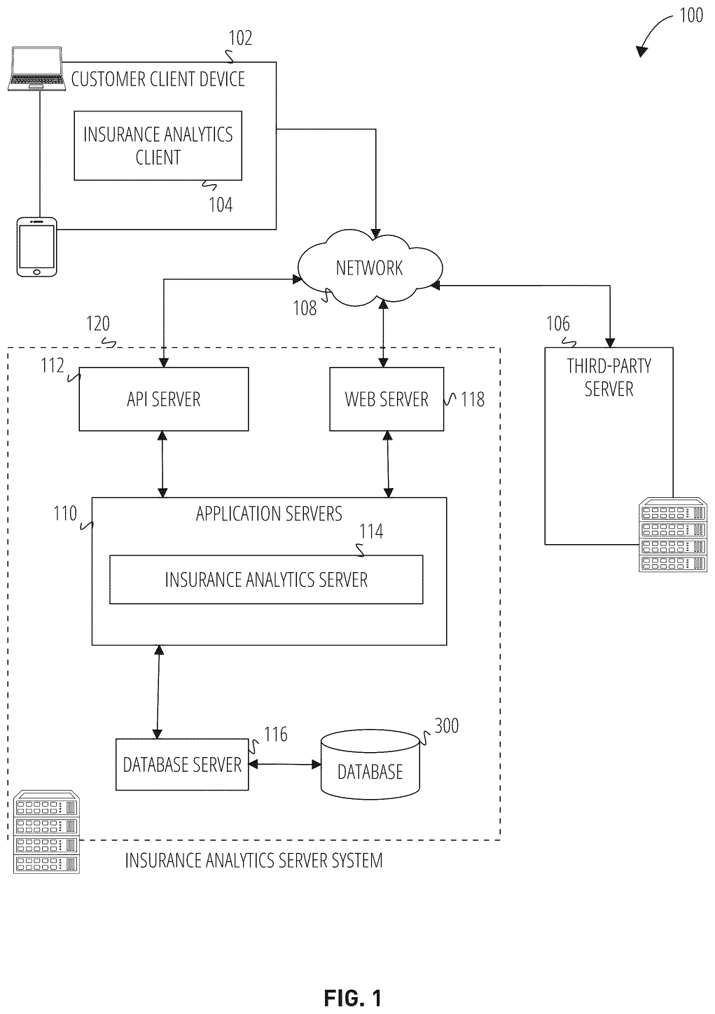

FIG. 1 is a block diagram showing an insurance analytics system 100 in accordance with some examples. The insurance analytics system 100 can include multiple instances of a customer client device 102 and multiple instances of a third-party server 106.

The customer client device 102 is associated with a client of the insurance analytics system 100. Examples of clients include financial institutions, insurance companies, analytics companies, etc. An underwriter (e.g., or administrative assistant, or other employee) can be the user of the customer client device 102.

Each of the customer client devices 102 hosts a number of applications, including an insurance analytics client 104. Each insurance analytics client 104 is communicatively coupled with an insurance analytics server system 120 and third-party servers 106 via a network 108 (e.g., communication network or the Internet). An insurance analytics client 104 can also communicate with locally-hosted applications using Applications Program Interfaces (APIs). The customer client devices 102 can also host a number of applications including Internet browsing applications (e.g., Chrome, Safari, etc.). The insurance analytics client 104 can also be implemented as a platform that is accessed by the customer client device 102 via an Internet browsing application or implemented as an extension on the Internet browsing application.

An insurance analytics client 104 is able to communicate and exchange data with the insurance analytics server system 120 via the network 108. The data exchanged between the insurance analytics client 104 and the insurance analytics server system 120, includes functions (e.g., commands to invoke functions) as well as payload data (e.g., underwriting manuals, risk submissions and applications, training material, feedback on the results and reporting provided).

The insurance analytics server system 120 can also communicate and exchange data with third-party server 106 to obtain further data and information on the customer, the applicants, as well as relevant standardized information (e.g., standardized codes). The third-party server 106 can be servers hosting different websites comprising this data and information.

The insurance analytics server system 120 supports various services and operations that are provided to the insurance analytics client 104. Such operations include access to the functionalities of the systems in insurance analytics server system 120. Data exchanges to and from the insurance analytics server system 120 are invoked and controlled through functions available via user interfaces (UIs) of the insurance analytics client 104.

The insurance analytics server system 120 provides server-side functionality via the network 108 to a particular insurance analytics client 104. While certain functions of the insurance analytics system 100 are described herein as being performed by either an insurance analytics client 104 or by the insurance analytics server system 120, the location of certain functionality either within the insurance analytics client 104 or the insurance analytics server system 120 may be a design choice. For example, it may be technically preferable to initially deploy certain technology and functionality within the insurance analytics server system 120 but to later migrate this technology and functionality to the insurance analytics client 104 where a customer client device 102 has sufficient processing capacity.

Turning now specifically to the insurance analytics server system 120, an Application Program Interface (API) server 112 is coupled to, and provides a programmatic interface to, application servers 110. The application servers 110 are communicatively coupled to a database server 116, which facilitates access to a database 300 that stores data from the third-party server 106 and customer client device 102 to be processed by the application servers 110. Similarly, a web server 118 is coupled to the application servers 110 and provides web-based interfaces to the application servers 110. To this end, the web server 118 processes incoming network requests over the Hypertext Transfer Protocol (HTTP) and several other related protocols.

The Application Program Interface (API) server 112 receives and transmits data between the customer client device 102 and the application servers 110. Specifically, the Application Program Interface (API) server 112 provides a set of interfaces (e.g., routines and protocols) that can be called or queried by the insurance analytics client 104 in order to invoke functionality of the application servers 110. The Application Program Interface (API) server 112 exposes to the insurance analytics client 104 various functions supported by the application servers 110, including generating information the risk evaluation of submissions, risk appetite result, inconsistency findings, etc.

The application servers 110 host a number of server applications and subsystems, including for example an insurance analytics server 114. The insurance analytics server 114 implements a number of data processing technologies and functions, particularly related to the processing of the customer's risk appetite, the risk analysis of submissions or applications, and the identification of inconsistencies in submissions requiring analysis of a plurality of sources. To perform these functions, the insurance analytics server 114 can also implement machine-learning solutions, neural networks, generative artificial intelligence (AI), natural language processing (NLP) techniques, etc. Other processor and memory intensive processing of data may also be performed server-side by the insurance analytics server 114, in view of the hardware requirements for such processing.

### SYSTEM ARCHITECTURE

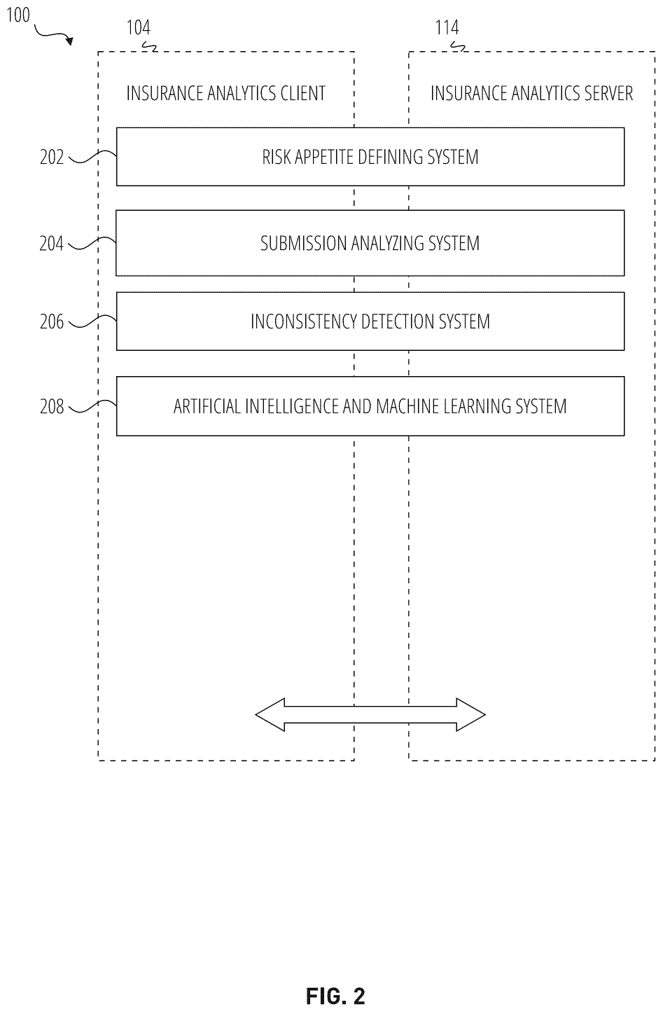

FIG. 2 is a block diagram illustrating further details regarding the insurance analytics system 100 according to some examples. Specifically, the insurance analytics system 100 is shown to comprise the insurance analytics client 104 and the insurance analytics server 114. The insurance analytics system 100 embodies a number of subsystems, which are supported on the client-side by the insurance analytics client 104 and on the server-side by the insurance analytics server 114. These subsystems include, for example, a risk appetite defining system 202, a submission analyzing system 204, an inconsistency detection system 206, and an artificial intelligence and machine learning system 208.

The risk appetite defining system 202 is responsible ingesting the customer's underwriting manual to extract rules that codify the customer's risk appetite. The risk appetite defining system 202 can receive the underwriting manual from the customer client device 102 or from the third-party server 106.

The submission analyzing system 204 is responsible for summarizing and classifying risk submissions that are received from the customer client device 102. The submission analyzing system 204 can further receive the risk appetite associated with a specific customer from the risk appetite defining system 202 to generate personalized recommendations regarding the risk associated with a given submission for a specific customer. The submission analyzing system 204 can further automatically classify the risk of the submission into standardized insurance codes by matching the summary description against classification databases in, for example, third-party server 106. Examples of classification codes assigned may include Standard Industrial Classification (SIC) codes and North American Industry Classification System (NAICS) codes.

The inconsistency detection system 206 is responsible for finding inconsistencies in data in different records. For example, the inconsistency detection system 206 can assess data from different medical records in differing formats including Portable Document Format (PDF), scanned images, Electronic Health Records (EHR), and Attending Physician Statement (APS) documents, etc. The data can be obtained by the inconsistency detection system 206 from third-party server 106 via the network 108 or from the customer client device 102. The inconsistency detection system 206 can further provide the inconsistency findings to the risk appetite defining system 202 to refine a determined risk classification. The inconsistency detection system 206 can also provide the inconsistency findings to the submission analyzing system 204 to further refine the submission analyzing system 204's summary and classification.

The artificial intelligence and machine learning system 208 provides a variety of services to different subsystems within the insurance analytics system 100. For example, the artificial intelligence and machine learning system 208 operates with the risk appetite defining system 202 to identify the language in the manual to be extracted and to generate the risk appetite rules and parameters based on this extracted language as well as the feedback received from the customer client device 102. The artificial intelligence and machine learning system 208 can also operate with risk appetite defining system 202 to generate the defined risk appetite for each of the customers. The artificial intelligence and machine learning system 208 can operate with the submission analyzing system 204 to generate the summaries and classifications based on the risk submissions that are received. The artificial intelligence and machine learning system 208 can also operate with the inconsistency detection system 206 to process the different sources of data to find the inconsistencies in the records.

### DATA ARCHITECTURE

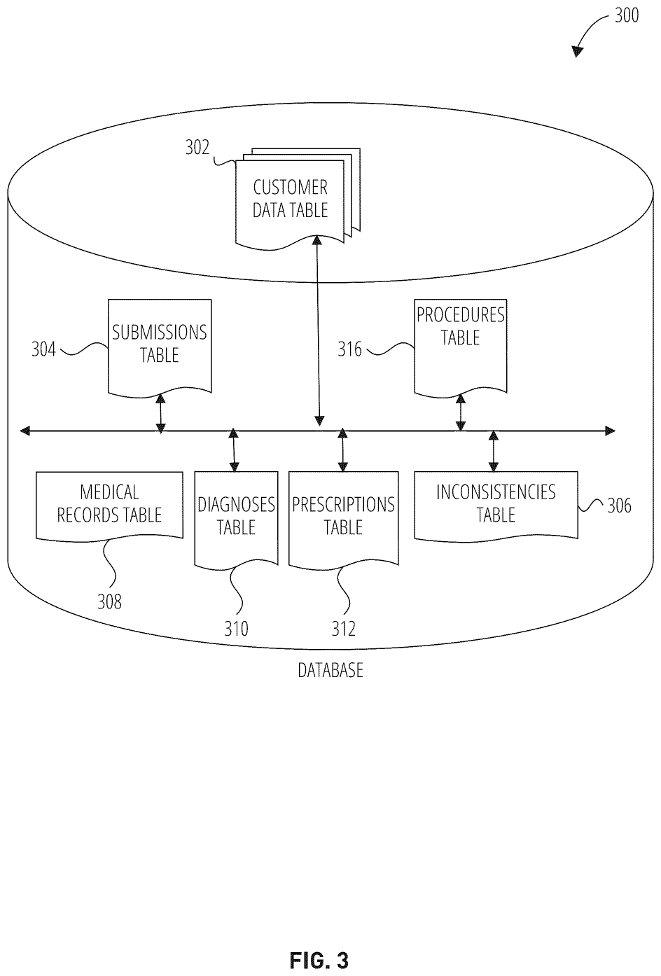

FIG. 3 is a schematic diagram illustrating database 300, which may be stored in the database 300 of the insurance analytics server 114, according to certain examples. While the content of the database 300 is shown to comprise a number of tables, it will be appreciated that the data could be stored in other types of data structures (e.g., as an object-oriented database).

The database 300 includes a customer data table 302, a submissions table 304, inconsistencies table 306, a medical records table 308, a diagnoses table 310, and a prescriptions table 312.

The customer data table 302 stores data related to the customers (or clients) of the insurance analytics system 100 including identification information, locations, business area, etc. The customer data table 302 also stores data including the underwriting manual for each of the customers, the extracted guidelines and rules from the manual, the risk appetite rules and parameters that are generated based on the underwriting manual and the feedback received from the customer client device 102, the defined risk appetite for each of the customers as codified by the risk appetite rules and parameters.

The submissions table 304 stores data related to the applications and submissions to be analyzed by the submission analyzing system 204. For example, the submissions table 304 stores the applications and submissions received from the customer client device 102 and any supporting documents that were provided with the applications and submissions. The submissions table 304 also stores additional information and data obtained from third-party server 106 including scraped website data and third-party data feeds that are relevant to the submission or application. The submissions table 304 also stores data related to the standardized insurance or classification codes that are obtained via training of the submission analyzing system 204 or from third-party server 106. The submissions table 304 can further store the standardized codes in association with the applications or submissions.

The inconsistencies table 306 stores the inconsistencies that are identified by the inconsistency detection system 206. The inconsistencies table 306 can further store trained machine learning models used to identify patterns of inconsistency based on semantic and textual features.

The medical records table 308 can store medical records in differing formats including Portable Document Format (PDF), scanned images, Electronic Health Records (EHR), and Attending Physician Statement (APS) documents. The medical records table 308 can further store documents where the inconsistencies are found.

The diagnoses table 310 can store medical diagnoses that are used to compare diagnoses data identified in the documents from the medical records table 308. The diagnoses table 310 can include diagnosed conditions retrieved from Attending Physicians Statements (APS), Electronic Health Records (EHR), and other third-party medical history databases. We use things like the following: Attending Physicians Statements (APS), Electronic Health Records (EHR), and other prescription and medical history record companies such as Milliman, MIB, and Irix which are provided by the carrier.

The prescriptions table 312 can store medical prescriptions that are used to compare the prescription data identified in the documents from the medical records table 308. The prescriptions table 312 can include a list of prescriptions retrieved from Attending Physicians Statements (APS), Electronic Health Records (EHR), and other third-party prescription databases.

The procedures table 316 can store a list of medical procedures that are used to compare historical medical procedures data identified in the documents from the medical records table 308. The procedures table 316 can include a list of medical procedures retrieved from Attending Physicians Statements (APS), Electronic Health Records (EHR), and other third-party medical history databases.

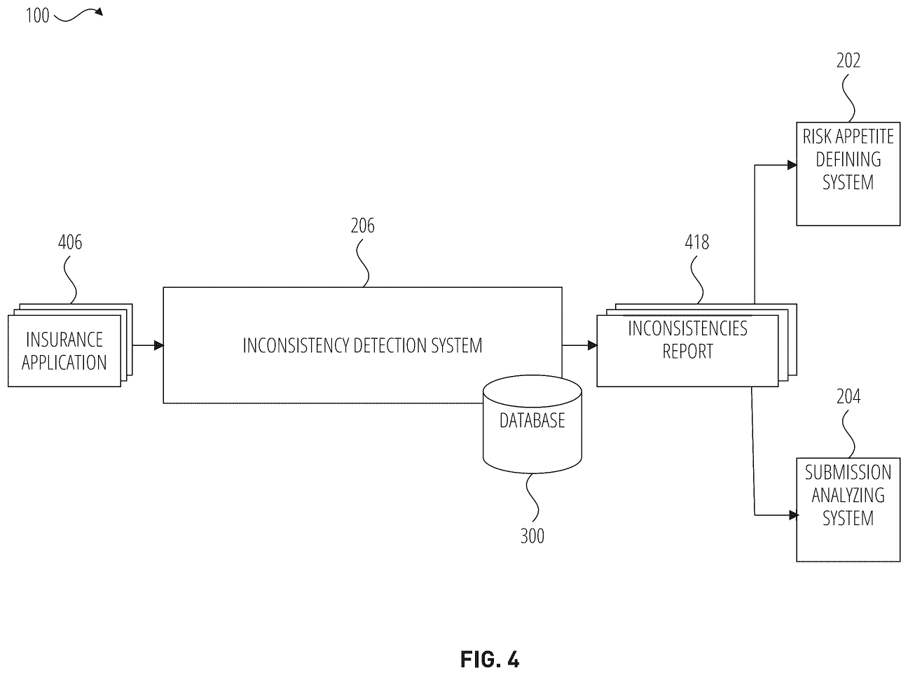

FIG. 4 illustrates the insurance analytics system 100 as being configured to identify inconsistencies in an insurance application, in accordance with some examples. For example, the identified inconsistencies are compiled into a report and can be used to analyze a risk appetite for an insurance carrier.

As shown in the example of FIG. 4, the insurance analytics system 100 includes the risk appetite defining system 202, the submission analyzing system 204 and the inconsistency detection system 206. While not shown in FIG. 4, the risk appetite defining system 202, the submission analyzing system 204, and the inconsistency detection system 206 can access or otherwise interact with the artificial intelligence and machine learning system 208 of FIG. 2.

The inconsistency detection system 206 is configured to analyze an insurance application 406 associated with a patient. The insurance application 406 can be provided by a customer client device 102 and stored within the customer data table 302 of the database 300. An insurance application 406 can be associated with multiple sources of medical records of a patient. The medical records can be accessed from the medical records table 308. The medical records contain both structured data and unstructured data.

The inconsistency detection system 206 generates inconsistencies report 418 which includes inconsistencies within a patient's insurance application 406 as pertaining to their medical records. The inconsistencies are identified based on comparing a patient's medical records to a database of prescription data and diagnoses data. The prescription data and the diagnoses data can be accessed from the prescriptions table 312 and the diagnoses table 310 respectively. The inconsistencies report 418 is subsequently displayed to a user of the insurance analytics system 100 on a user interface of a computer device. In some examples, the inconsistencies table 306 is provided as an input to the risk appetite defining system 202 and the submission analyzing system 204.

The inconsistency detection system 206 operates with the artificial intelligence and machine learning system 208 to identify the data in the insurance application 406 to be extracted, and to generate the inconsistencies report 418.

The artificial intelligence and machine learning system 208 accesses machine learning models including natural language processing (NLP) models and optical character recognition (OCR) models. The NLP and OCR models are used to extract relevant data from an insurance application 406 and associated medical records of a patient and analyze the data using machine learning based information extraction techniques including named entity recognition, relation extraction and coreference resolution. The NLP and OCR models can be pre-trained models that are trained on labeled medical records datasets.

The extracted data can be compared against a database of medical data (e.g., database 300) using graph similarity metrics and rule-based logic to identify inconsistencies.

The artificial intelligence and machine learning system 208 further accesses neural network models in order to determine the inconsistencies based on the insurance application 406 based on the extracted data by the NLP and OCR models. The neural network models include one or more large language models (LLM) and corresponds to transformer-based models which are fine-tuned on insurance data to understand the complex language and terminology in underwriting manuals and insurance applications. The LLMs are trained on medical databases, medical records, insurance applications, and relevant patient data including but not limited to driving records and lifestyle data.

For example, the artificial intelligence and machine learning system 208 includes one or more generative pre-trained transformers (e.g., OpenAI GPT, Anthropic Claude, and the like) in combination with additional models pre-trained on medical data for identifying inconsistencies in an insurance application. The identified inconsistencies are thus further analyzed by the LLMs to identify patterns based on semantic and textual features of the extracted data and rationalize the identified inconsistencies.

### PROCESS OF IDENTIFYING INCONSISTENCIES

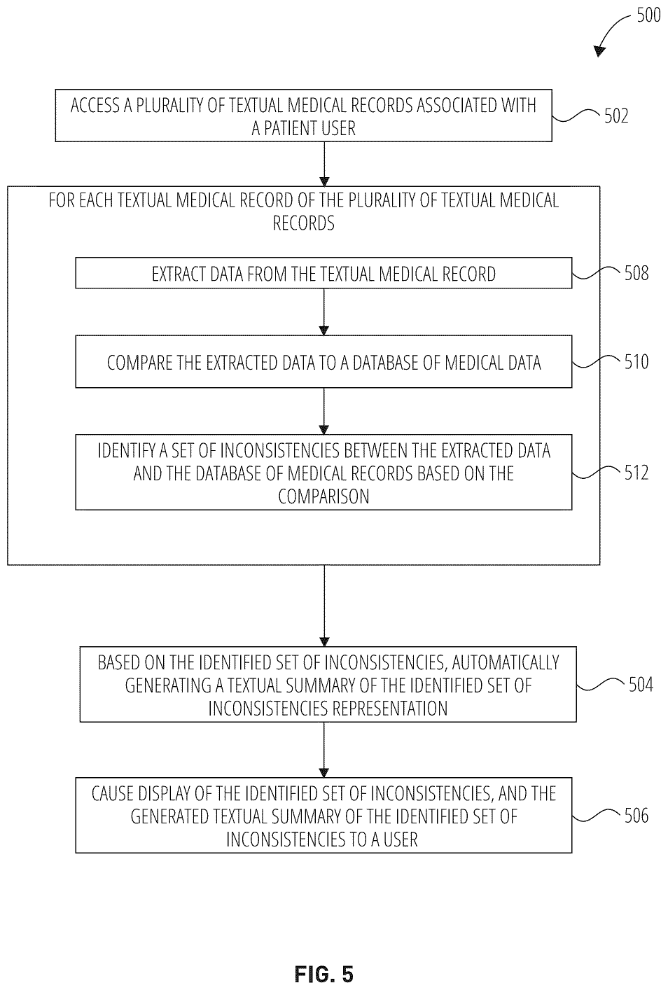

FIG. 5 is a flowchart illustrating a process 500 for identifying inconsistencies in an insurance application, in accordance with some examples. For explanatory purposes, the process 600 is primarily described herein with reference to the risk appetite defining system 202, submission analyzing system 204, and the inconsistency detection system 206 of FIG. 2, and the customer client device 102 of FIG. 1. However, one or more blocks (or operations) of the process 500 may be performed by one or more other components, and/or by other suitable devices. Further for explanatory purposes, the blocks (or operations) of the process 500 are described herein as occurring in serial, or linearly. However, multiple blocks (or operations) of the process 500 may occur in parallel or concurrently. In addition, the blocks (or operations) of the process 500 need not be performed in the order shown and/or one or more blocks (or operations) of the process 500 need not be performed and/or can be replaced by other operations. The process 500 may be terminated when its operations are completed. In addition, the process 500 may correspond to a method, a procedure, an algorithm, etc.

At operation 512, the inconsistency detection system 206 accesses a plurality of textual medical records associated with a patient user. The textual medical records can be accessed from the medical records table 308. The plurality of textual records can be accessed in response to receiving an insurance application (e.g., insurance application 406) from a patient user for underwriting. In some examples, each textual medical record is associated with a weight value indicating an importance of the textual medical record.

For each textual medical record of the plurality of textual medical records, the inconsistency detection system 206 at operation 508 extracts data from the textual medical record, at operation 510, compares the extracted data to a database of medical data, and at operation 512 identifies a set of inconsistencies between the extracted data and the database of medical records based on the comparison. The extracted data in operation 508 can be medical data that is extracted using the OCR models and NLP models described above in connection with FIG. 4. The database of medical data can include data in the customer data table 302, the diagnoses table 310, the prescriptions table 312, and the procedures table 316. The identified set of inconsistencies are related to insurance underwriting decisions associated with the insurance application of the patient user.

At operation 504, based on the identified set of inconsistencies, the inconsistency detection system 206 automatically generates a textual summary of the identified set of inconsistencies. The textual summary of the identified set of inconsistencies is generated using large language models as described above in connection with FIG. 4. In some examples, the weight value of each textual medical record is further provided as input to the large language model. The weight value may be a value between zero and one. If a document with higher importance (e.g., a higher weight value) is found to have a discrepancy or inconsistency with a document with lower importance (e.g., a lower weight value), the inconsistency detection system 206 may automatically resolve the inconsistency by prioritizing the data in the document with higher importance. In another example, if the document with the higher importance is found to have an inconsistency with a document with lower importance, the generated textual summary can include a textual rationalization of the discrepancy based on the differing weight values of the analyzed documents.

At operation 506, the inconsistency detection system 206 causes display of the identified set of inconsistencies, and the generated textual summary of the identified set of inconsistencies to a user. The generated textual summaries rationalize the identified discrepancies in a humanlike conversational form. In some examples, the identified set of inconsistencies are displayed in an order of importance. The order may be based on a weight of the textual medical records that were analyzed. In some examples, an operator of the inconsistency detection system 206 can flag categories of data that are high priority. If inconsistencies are detected in the flagged categories of data, such inconsistencies can be marked as having a higher level of importance.

In some examples, the identified set of inconsistencies is provided as input to at least one of the risk appetite defining system 202 and the submission analyzing system 204. In some examples, the identified set of inconsistencies is used to generate a flag value associated with the insurance application of the patient user. The flag value can subsequently be provided to at least one of the risk appetite defining system 202 and the submission analyzing system 204. In some examples, the identified set of inconsistences is used to validate against the risk appetite defining system 202 and surfaces the inconsistencies to a user of the risk appetite defining system.

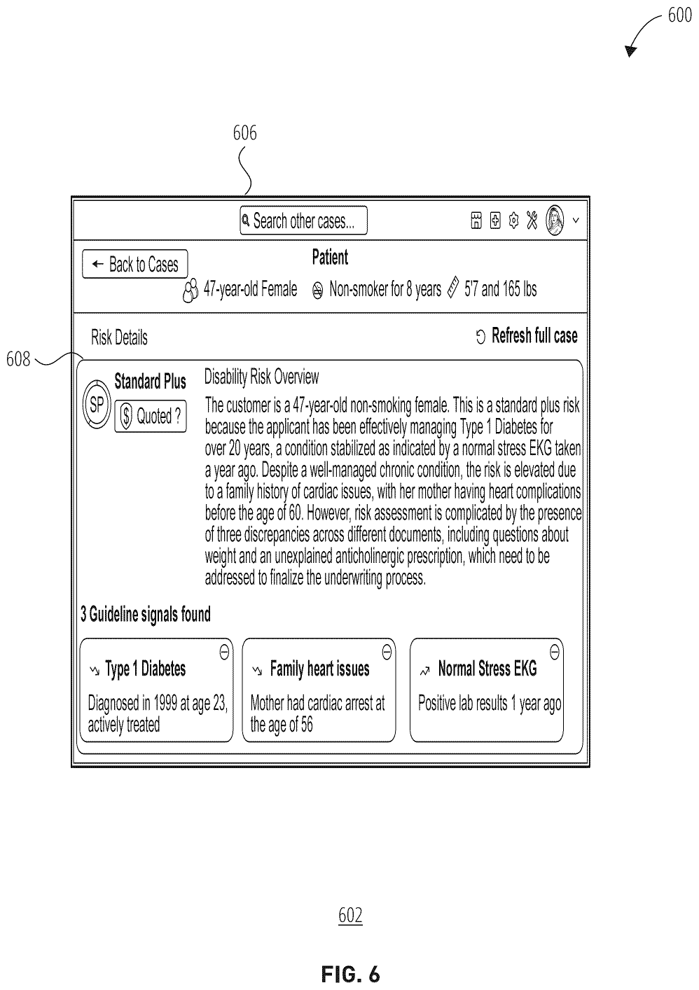

FIG. 6 illustrates examples of the inconsistency detection system 206 interface in accordance with one embodiment. The inconsistency detection system 206 generates inconsistencies report 606. The report 606 is shown to include a risk overview 608 of a patient and highlights discrepancies and inconsistencies in the patient's insurance application and associated medical records. The risk overview 608 is provided in a humanlike conversational form by providing context for the identified inconsistencies which thereby improves efficiency of the underwriting process.

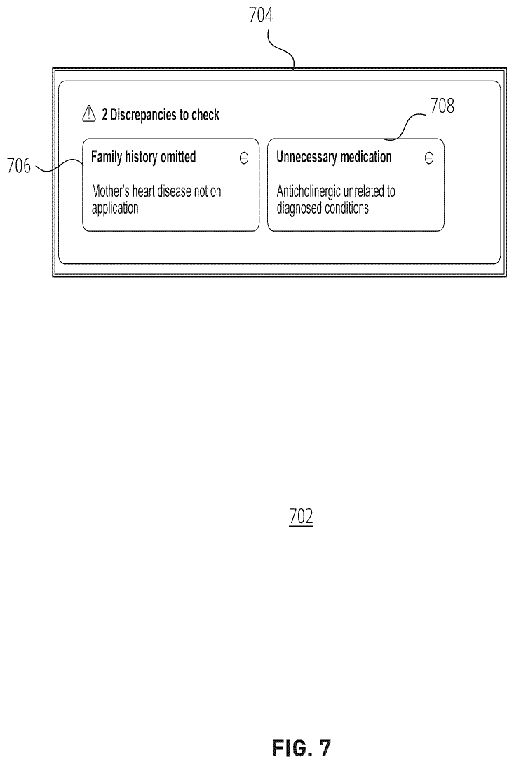

FIG. 7 illustrates examples of the inconsistency detection system 206 interface in accordance with one embodiment. The inconsistencies report 606 can further include a user interface component 704 which details the identified discrepancies and inconsistencies. The component 704 further includes user interface elements 706 and 708 which further explain the identified inconsistencies. The text generated in the user interface elements 706 and 708 can be generated using the large language models described above in connection with FIG. 4. In some examples the user interface elements 706 and 708 are selectable elements. For example, selection of elements 706 and 708 can prompt the inconsistency detection system 206 to display and highlight portions of the relevant medical documents where the inconsistencies were identified.

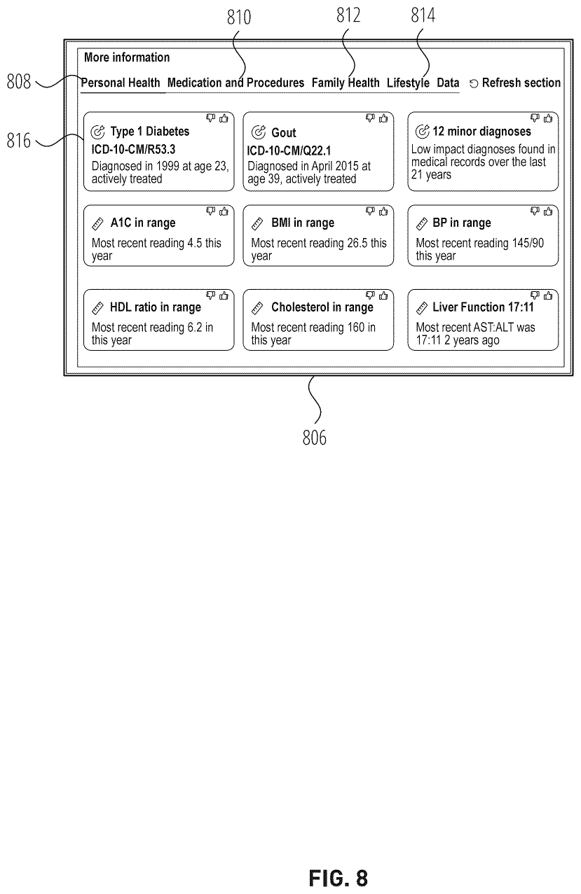

FIG. 8 illustrates examples of the inconsistency detection system 206 interface in accordance with one embodiment. The inconsistencies report 606 can further include a user interface component 806 which highlights aspects of the patient's insurance application. The user interface component 806 can include scrollable elements 808, 810, 812, and 814 which upon selection, each cause display of highlights relating to the appropriate category of medical information described within the text of the scrollable elements 808, 810, 812 and 814. For example, the text displayed within the highlight 816 can be generated using a large language model as described above in connection with FIG. 4.

### MACHINE ARCHITECTURE

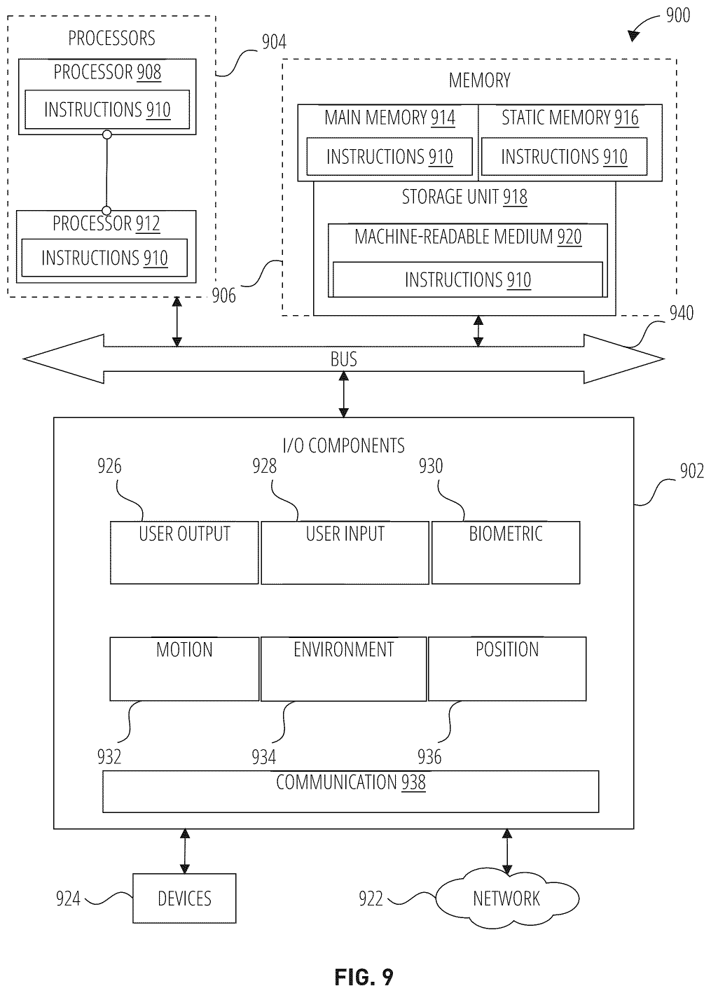

In some examples, components in the insurance analytics system 100 can be a machine 900 as shown in FIG. 9. FIG. 9 is a diagrammatic representation of the machine 900 within which instructions 910 (e.g., software, a program, an application, an applet, an application, or other executable code) for causing the machine 900 to perform any one or more of the methodologies discussed herein may be executed. For example, the instructions 910 may cause the machine 900 to execute any one or more of the methods described herein. The instructions 910 transform the general, non-programmed machine 900 into a particular machine 900 programmed to carry out the described and illustrated functions in the manner described. The machine 900 may operate as a standalone device or may be coupled (e.g., networked) to other machines. In a networked deployment, the machine 900 may operate in the capacity of a server machine or a client machine in a server-client network environment, or as a peer machine in a peer-to-peer (or distributed) network environment. The machine 900 may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smartphone, a mobile device, a wearable device (e.g., a smartwatch), a smart home device (e.g., a smart appliance), other smart devices, a web appliance, a network router, a network switch, a network bridge, or any machine capable of executing the instructions 910, sequentially or otherwise, that specify actions to be taken by the machine 900. Further, while only a single machine 900 is illustrated, the term “machine” shall also be taken to include a collection of machines that individually or jointly execute the instructions 910 to perform any one or more of the methodologies discussed herein. The machine 900, for example, may comprise the customer client device 102 or any one of a number of server devices forming part of the insurance analytics server 114. In some examples, the machine 900 may also comprise both client and server systems, with certain operations of a particular method or algorithm being performed on the server-side and with certain operations of the particular method or algorithm being performed on the client-side.

The machine 900 may include processors 904, memory 906, and input/output I/O components 902, which may be configured to communicate with each other via a bus 940. In an example, the processors 904 (e.g., a Central Processing Unit (CPU), a Reduced Instruction Set Computing (RISC) Processor, a Complex Instruction Set Computing (CISC) Processor, a Graphics Processing Unit (GPU), a Digital Signal Processor (DSP), an Application Specific Integrated Circuit (ASIC), a Radio-Frequency Integrated Circuit (RFIC), another processor, or any suitable combination thereof) may include, for example, a processor 908 and a processor 912 that execute the instructions 910. The term “processor” is intended to include multi-core processors that may comprise two or more independent processors (sometimes referred to as “cores”) that may execute instructions contemporaneously. Although FIG. 9 shows multiple processors 904, the machine 900 may include a single processor with a single-core, a single processor with multiple cores (e.g., a multi-core processor), multiple processors with a single core, multiple processors with multiples cores, or any combination thereof.

The memory 906 includes a main memory 914, a static memory 916, and a storage unit 918, both accessible to the processors 904 via the bus 940. The main memory 914, the static memory 916, and storage unit 918 store the instructions 910 embodying any one or more of the methodologies or functions described herein. The instructions 910 may also reside, completely or partially, within the main memory 914, within the static memory 916, within machine-readable medium 920 within the storage unit 918, within at least one of the processors 904 (e.g., within the processor's cache memory), or any suitable combination thereof, during execution thereof by the machine 900.

The I/O components 902 may include a wide variety of components to receive input, provide output, produce output, transmit information, exchange information, capture measurements, and so on. The specific I/O components 902 that are included in a particular machine will depend on the type of machine. For example, portable machines such as mobile phones may include a touch input device or other such input mechanisms, while a headless server machine will likely not include such a touch input device. It will be appreciated that the I/O components 902 may include many other components that are not shown in FIG. 9. In various examples, the I/O components 902 may include user output components 926 and user input components 928. The user output components 926 may include visual components (e.g., a display such as a plasma display panel (PDP), a light-emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The user input components 928 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and force of touches or touch gestures, or other tactile input components), audio input components (e.g., a microphone), and the like.

In further examples, the I/O components 902 may include biometric components 930, motion components 932, environmental components 934, or position components 936, among a wide array of other components. For example, the biometric components 930 include components to detect expressions (e.g., hand expressions, facial expressions, vocal expressions, body gestures, or eye-tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 932 include acceleration sensor components (e.g., accelerometer), gravitation sensor components, rotation sensor components (e.g., gyroscope).

The environmental components 934 include, for example, one or cameras (with still image/photograph and video capabilities), illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment.

With respect to cameras, the customer client device 102 may have a camera system comprising, for example, front cameras on a front surface of the customer client device 102 and rear cameras on a rear surface of the customer client device 102. The front cameras may, for example, be used to capture still images and video of a user of the customer client device 102 (e.g., “selfies”). The rear cameras may, for example, be used to capture still images and videos in a more traditional camera mode. In addition to front and rear cameras, the customer client device 102 may also include a 360° camera for capturing 360° photographs and videos.

Further, the camera system of a customer client device 102 may include dual rear cameras (e.g., a primary camera as well as a depth-sensing camera), or even triple, quad or penta rear camera configurations on the front and rear sides of the customer client device 102. These multiple cameras systems may include a wide camera, an ultra-wide camera, a telephoto camera, a macro camera and a depth sensor, for example.

The position components 936 include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 902 further include communication components 938 operable to couple the machine 900 to a network 922 or devices 924 via respective coupling or connections. For example, the communication components 938 may include a network interface component or another suitable device to interface with the network 922. In further examples, the communication components 938 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 924 may be another machine or any of a wide variety of peripheral devices (e.g., a peripheral device coupled via a USB).

Moreover, the communication components 938 may detect identifiers or include components operable to detect identifiers. For example, the communication components 938 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 938, such as location via Internet Protocol (IP) geolocation, location via Wi-Fi® signal triangulation, location via detecting an NFC beacon signal that may indicate a particular location, and so forth.

The various memories (e.g., main memory 914, static memory 916, and memory of the processors 904) and storage unit 918 may store one or more sets of instructions and data structures (e.g., software) embodying or used by any one or more of the methodologies or functions described herein. These instructions (e.g., the instructions 910), when executed by processors 904, cause various operations to implement the disclosed examples.

The instructions 910 may be transmitted or received over the network 922, using a transmission medium, via a network interface device (e.g., a network interface component included in the communication components 938) and using any one of several well-known transfer protocols (e.g., hypertext transfer protocol (HTTP)). Similarly, the instructions 910 may be transmitted or received using a transmission medium via a coupling (e.g., a peer-to-peer coupling) to the devices 924.

### SOFTWARE ARCHITECTURE

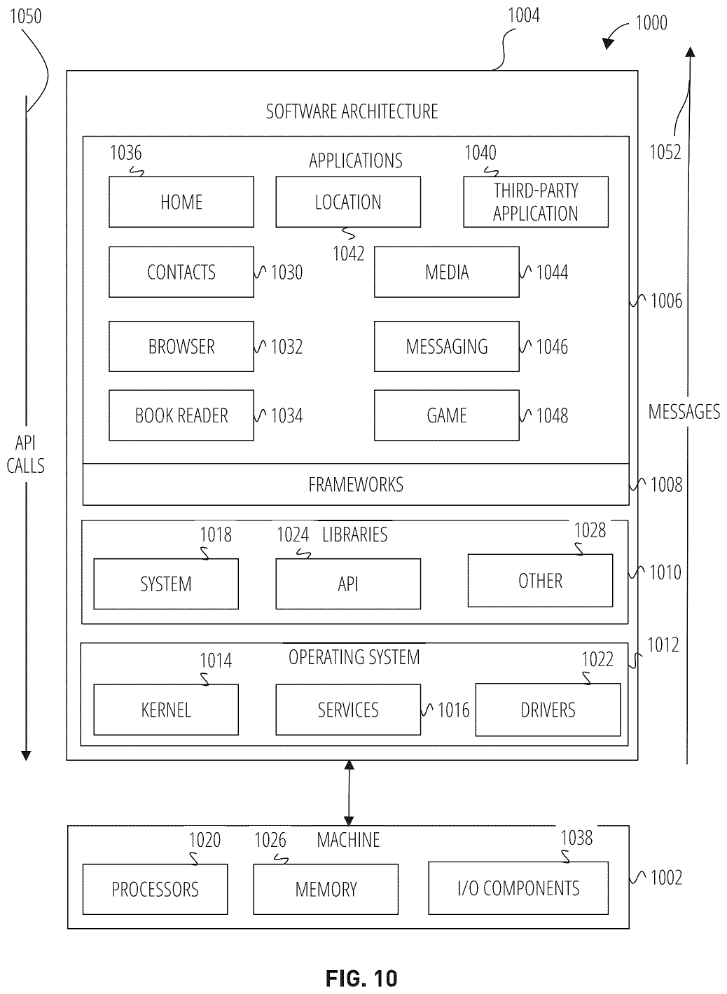

FIG. 10 is a block diagram 1000 illustrating a software architecture 1004, which can be installed on any one or more of the devices described herein. The software architecture 1004 is supported by hardware such as a machine 1002 that includes processors 1020, memory 1026, and I/O components 1038. In this example, the software architecture 1004 can be conceptualized as a stack of layers, where each layer provides a particular functionality. The software architecture 1004 includes layers such as an operating system 1012, libraries 1010, frameworks 1008, and applications 1006. Operationally, the applications 1006 invoke API calls 1050 through the software stack and receive messages 1052 in response to the API calls 1050.

The operating system 1012 manages hardware resources and provides common services. The operating system 1012 includes, for example, a kernel 1014, services 1016, and drivers 1022. The kernel 1014 acts as an abstraction layer between the hardware and the other software layers. For example, the kernel 1014 provides memory management, processor management (e.g., scheduling), component management, networking, and security settings, among other functionalities. The services 1016 can provide other common services for the other software layers. The drivers 1022 are responsible for controlling or interfacing with the underlying hardware. For instance, the drivers 1022 can include display drivers, camera drivers, BLUETOOTH® or BLUETOOTH® Low Energy drivers, flash memory drivers, serial communication drivers (e.g., USB drivers), WI-FI® drivers, audio drivers, power management drivers, and so forth.

The libraries 1010 provide a common low-level infrastructure used by the applications 1006. The libraries 1010 can include system libraries 1018 (e.g., C standard library) that provide functions such as memory allocation functions, string manipulation functions, mathematic functions, and the like. In addition, the libraries 1010 can include API libraries 1024 such as media libraries (e.g., libraries to support presentation and manipulation of various media formats such as Moving Picture Experts Group-4 (MPEG4), Advanced Video Coding (H.264 or AVC), Moving Picture Experts Group Layer-3 (MP3), Advanced Audio Coding (AAC), Adaptive Multi-Rate (AMR) audio codec, Joint Photographic Experts Group (JPEG or JPG), or Portable Network Graphics (PNG)), graphics libraries (e.g., an OpenGL framework used to render in two dimensions (2D) and three dimensions (3D) in a graphic content on a display), database libraries (e.g., SQLite to provide various relational database functions), web libraries (e.g., WebKit to provide web browsing functionality), and the like. The libraries 1010 can also include a wide variety of other libraries 1028 to provide many other APIs to the applications 1006.

The frameworks 1008 provide a common high-level infrastructure that is used by the applications 1006. For example, the frameworks 1008 provide various graphical user interface (GUI) functions, high-level resource management, and high-level location services. The frameworks 1008 can provide a broad spectrum of other APIs that can be used by the applications 1006, some of which may be specific to a particular operating system or platform.

In an example, the applications 1006 may include a home application 1036, a contacts application 1030, a browser application 1032, a book reader application 1034, a location application 1042, a media application 1044, a messaging application 1046, a game application 1048, and a broad assortment of other applications such as a third-party application 1040. The applications 1006 are programs that execute functions defined in the programs. Various programming languages can be employed to create one or more of the applications 1006, structured in a variety of manners, such as object-oriented programming languages (e.g., Objective-C, Java, or C++) or procedural programming languages (e.g., C or assembly language). In a specific example, the third-party application 1040 (e.g., an application developed using the ANDROID™ or IOS™ software development kit (SDK) by an entity other than the vendor of the particular platform) may be mobile software running on a mobile operating system such as IOS™, ANDROID™, WINDOWS® Phone, or another mobile operating system. In this example, the third-party application 1040 can invoke the API calls 1050 provided by the operating system 1012 to facilitate functionality described herein.

### GLOSSARY

“Carrier signal” refers to any intangible medium that is capable of storing, encoding, or carrying instructions for execution by the machine, and includes digital or analog communications signals or other intangible media to facilitate communication of such instructions. Instructions may be transmitted or received over a network using a transmission medium via a network interface device.

“Client device” refers to any machine that interfaces to a communications network to obtain resources from one or more server systems or other client devices. A client device may be, but is not limited to, a mobile phone, desktop computer, laptop, portable digital assistants (PDAs), smartphones, tablets, ultrabooks, netbooks, laptops, multi-processor systems, microprocessor-based or programmable consumer electronics, game consoles, set-top boxes, or any other communication device that a user may use to access a network.

“Communication network” refers to one or more portions of a network that may be an ad hoc network, an intranet, an extranet, a virtual private network (VPN), a local area network (LAN), a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network and the coupling may be a Code Division Multiple Access (CDMA) connection, a Global System for Mobile communications (GSM) connection, or other types of cellular or wireless coupling. In this example, the coupling may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long-range protocols, or other data transfer technology.

“Component” refers to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various examples, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein. A hardware component may also be implemented mechanically, electronically, or any suitable combination thereof. For example, a hardware component may include dedicated circuitry or logic that is permanently configured to perform certain operations. A hardware component may be a special-purpose processor, such as a field-programmable gate array (FPGA) or an application specific integrated circuit (ASIC). A hardware component may also include programmable logic or circuitry that is temporarily configured by software to perform certain operations. For example, a hardware component may include software executed by a general-purpose processor or other programmable processor. Once configured by such software, hardware components become specific machines (or specific components of a machine) uniquely tailored to perform the configured functions and are no longer general-purpose processors. It will be appreciated that the decision to implement a hardware component mechanically, in dedicated and permanently configured circuitry, or in temporarily configured circuitry (e.g., configured by software), may be driven by cost and time considerations. Accordingly, the phrase “hardware component” (or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering examples in which hardware components are temporarily configured (e.g., programmed), each of the hardware components need not be configured or instantiated at any one instance in time. For example, where a hardware component comprises a general-purpose processor configured by software to become a special-purpose processor, the general-purpose processor may be configured as respectively different special-purpose processors (e.g., comprising different hardware components) at different times. Software accordingly configures a particular processor or processors, for example, to constitute a particular hardware component at one instance of time and to constitute a different hardware component at a different instance of time. Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In examples in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output. Hardware components may also initiate communications with input or output devices, and can operate on a resource (e.g., a collection of information). The various operations of example methods described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions described herein. As used herein, “processor-implemented component” refers to a hardware component implemented using one or more processors. Similarly, the methods described herein may be at least partially processor-implemented, with a particular processor or processors being an example of hardware. For example, at least some of the operations of a method may be performed by one or more processors or processor-implemented components. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an API). The performance of certain of the operations may be distributed among the processors, not only residing within a single machine, but deployed across a number of machines. In some examples, the processors or processor-implemented components may be located in a single geographic location (e.g., within a home environment, an office environment, or a server farm). In other examples, the processors or processor-implemented components may be distributed across a number of geographic locations.

“Computer-readable storage medium” refers to both machine-storage media and transmission media. Thus, the terms include both storage devices/media and carrier waves/modulated data signals. The terms “machine-readable medium,” “computer-readable medium” and “device-readable medium” mean the same thing and may be used interchangeably in this disclosure.

“Machine storage medium” refers to a single or multiple storage devices and media (e.g., a centralized or distributed database, and associated caches and servers) that store executable instructions, routines and data. The term shall accordingly be taken to include, but not be limited to, solid-state memories, and optical and magnetic media, including memory internal or external to processors. Specific examples of machine-storage media, computer-storage media and device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks The terms “machine-storage medium,” “device-storage medium,” “computer-storage medium” mean the same thing and may be used interchangeably in this disclosure. The terms “machine-storage media,” “computer-storage media,” and “device-storage media” specifically exclude carrier waves, modulated data signals, and other such media, at least some of which are covered under the term “signal medium.”

“Non-transitory computer-readable storage medium” refers to a tangible medium that is capable of storing, encoding, or carrying the instructions for execution by a machine.

“Signal medium” refers to any intangible medium that is capable of storing, encoding, or carrying the instructions for execution by a machine and includes digital or analog communications signals or other intangible media to facilitate communication of software or data. The term “signal medium” shall be taken to include any form of a modulated data signal, carrier wave, and so forth. The term “modulated data signal” means a signal that has one or more of its characteristics set or changed in such a matter as to encode information in the signal. The terms “transmission medium” and “signal medium” mean the same thing and may be used interchangeably in this disclosure.

1. A method comprising:
accessing a plurality of textual medical records associated with a patient user;
training a machine learning model to identify inconsistencies in the plurality of textual medical records using a database comprising unstructured attending physician statement documents and structured electronic health records retrieved from a set of medical history databases;
accessing a trained large language model (LLM);
fine-tuning the LLM using a medical database to understand medical terminology in the plurality of textual medical records;
for each textual medical record of the plurality of textual medical records:
extracting data from the textual medical record; and
comparing the extracted data to a database of medical data using the machine learning model to identify inconsistencies and the LLM to automatically resolve the inconsistencies based on weight values associated with the extracted data and the database of medical data;
identifying a set of inconsistencies between the extracted data and the database of medical data that are not automatically resolved by the LLM;
based on the identified set of inconsistencies, automatically generating a textual summary of the identified set of inconsistencies using the LLM;
based on an application of the patient user, automatically generating text summaries of the application corresponding with categories of patent information in the application using the LLM;
causing display of an interface comprising a set of selectable elements corresponding with the identified set of inconsistencies, a set of scrollable elements corresponding with the categories of patient information, and the generated textual summary of the identified set of inconsistencies, each scrollable element displaying a scrollable text summary of a corresponding category of patient information generated using the LLM;
in response to a first selection of a first selectable element of the set of selectable elements, causing display of a first textual medical record with a first highlighted portion and a second textual medical record with a second highlighted portion in the interface, the first highlighted portion and the second highlighted portion corresponding with a first identified inconsistency of the identified set of inconsistencies, the first identified inconsistency corresponding with the first selectable element; and
in response to a second selection of a first scrollable element of the set of scrollable elements, causing display of a highlighted portion of the application related to a first category of patient information, the first category of patient information corresponding with the first scrollable element.

2. The method of claim 1, wherein the plurality of textual medical records comprise structured data and unstructured data.

3. The method of claim 1, wherein the plurality of textual medical records are accessed from a medical records table based on the application of the patient user.

4. The method of claim 1, wherein the identified set of inconsistencies are related to insurance underwriting decisions associated with the application of the patient user.

5. The method of claim 1, wherein the extracted data comprises textual medical data, the method further comprising:
extracting the medical data from the textual medical record using a trained natural language processing machine learning model or a trained optical character recognition machine learning model trained to analyze the textual medical record.

6. The method of claim 1, wherein the first scrollable element of the set of scrollable elements displays a first scrollable text summary of the first category of patient information, the first scrollable text summary generated using the LLM based on the highlighted portion of the application.

7. The method of claim 1, wherein each textual medical record is associated with a weight value indicating an importance of the textual medical record, the method further comprising:
automatically resolving a discrepancy between a third textual medical record and a fourth textual medical record by prioritizing the third textual medical record based on a first weight value associated with the third textual medical record being higher than a second weight value associated with the fourth textual medical record.

8. A system comprising:
a processor; and
a memory storing instructions that, when executed by the processor, cause the system to perform operations comprising:
accessing a plurality of textual medical records associated with a patient user;
training a machine learning model to identify inconsistencies in the plurality of textual medical records using a database comprising unstructured attending physician statement documents and structured electronic health records retrieved from a set of medical history databases;
accessing a trained large language model (LLM);
fine-tuning the LLM using a medical database to understand medical terminology in the plurality of textual medical records;
for each textual medical record of the plurality of textual medical records;
extracting data from the textual medical record; and
comparing the extracted data to a database of medical data using the machine learning model to identify inconsistencies and the LLM to automatically resolve the inconsistencies based on weight values associated with the extracted data and the database of medical data:
identifying a set of inconsistencies between the extracted data and the database of medical data that are not automatically resolved by the LLM;
based on the identified set of inconsistencies, automatically generating a textual summary of the identified set of inconsistencies using the LLM;
based on an application of the patient user, automatically generating text summaries of the application corresponding with categories of patent information in the application using the LLM;
causing display of an interface comprising a set of selectable elements corresponding with the identified set of inconsistencies, a set of scrollable elements corresponding with the categories of patient information, and the generated textual summary of the identified set of inconsistencies, each scrollable element displaying a scrollable text summary of a corresponding category of patient information generated using the LLM;
in response to a first selection of a first selectable element of the set of selectable elements, causing display of a first textual medical record with a first highlighted portion and a second textual medical record with a second highlighted portion in the interface, the first highlighted portion and the second highlighted portion corresponding with a first identified inconsistency of the identified set of inconsistencies, the first identified inconsistency corresponding with the first selectable element; and
in response to a second selection of a first scrollable element of the set of scrollable elements, causing display of a highlighted portion of the application related to a first category of patient information, the first category of patient information corresponding with the first scrollable element.

9. The system of claim 8, wherein the plurality of textual medical records comprise structured data and unstructured data.

10. The system of claim 8, wherein the plurality of textual medical records are accessed from a medical records table based on the application of the patient user.

11. The system of claim 8, wherein the identified set of inconsistencies are related to insurance underwriting decisions associated with the application of the patient user.

12. The system of claim 8, wherein the extracted data comprises textual medical data, the operations further comprising:
extracting the textual medical data from the textual medical record using a trained natural language processing machine learning model or a trained optical character recognition machine learning model trained to analyze the textual medical record.

13. The system of claim 8, wherein the first scrollable element of the set of scrollable elements displays a first scrollable text summary of the first category of patient information, the first scrollable text summary generated using the LLM based on the highlighted portion of the application.

14. The system of claim 8, wherein each textual medical record is associated with a weight value indicating an importance of the textual medical record.

15. A non-transitory computer-readable storage medium including instructions that when executed by a processor, cause the processor to perform operations comprising:
accessing a plurality of textual medical records associated with a patient user;
training a machine learning model to identify inconsistencies in the plurality of textual medical records using a database comprising unstructured attending physician statement documents and structured electronic health records retrieved from a set of medical history databases;
accessing a trained large language model (LLM);
fine-tuning the LLM using a medical database to understand medical terminology in the plurality of textual medical records;
for each textual medical record of the plurality of textual medical records:
extracting data from the textual medical record; and
comparing the extracted data to a database of medical data using the machine learning model to identify inconsistencies and the LLM to automatically resolve the inconsistencies based on weight values associated with the extracted data and the database of medical data;
identifying a set of inconsistencies between the extracted data and the database of medical data that are not automatically resolved by the LLM. based on the identified set of inconsistencies, automatically generating a textual summary of the identified set of inconsistencies using the LLM;
based on an application of the patient user, automatically generating text summaries of the application corresponding with categories of patent information in the application using the LLM;
causing display of an interface comprising a set of selectable elements corresponding with the identified set of inconsistencies, a set of scrollable elements corresponding with the categories of patient information, and the generated textual summary of the identified set of inconsistencies, each scrollable element displaying a scrollable text summary of a corresponding category of patient information generated using the LLM;
in response to a first selection of a first selectable element of the set of selectable elements, causing display of a first textual medical record with a first highlighted portion and a second textual medical record with a second highlighted portion in the interface, the first highlighted portion and the second highlighted portion corresponding with a first identified inconsistency of the identified set of inconsistencies, the first identified inconsistency corresponding with the first selectable element; and
in response to a second selection of a first scrollable element of the set of scrollable elements, causing display of a highlighted portion of the application related to a first category of patient information, the first category of patient information corresponding with the first scrollable element.

16. The non-transitory computer-readable storage medium of claim 15, wherein the plurality of textual medical records comprise structured data and unstructured data.

17. The non-transitory computer-readable storage medium of claim 15, wherein the plurality of textual medical records are accessed from a medical records table based on the application of the patient user.

18. The non-transitory computer-readable storage medium of claim 15, wherein the identified set of inconsistencies are related to insurance underwriting decisions associated with the application of the patient user.

19. The non-transitory computer-readable storage medium of claim 15, wherein the extracted data comprises textual medical data, the operations further comprising:
extracting the medical data from the textual medical record using a trained natural language processing machine learning model or a trained optical character recognition machine learning model trained to analyze the textual medical record.

20. The non-transitory computer-readable storage medium of claim 15, wherein the first scrollable element of the set of scrollable elements displays a first scrollable text summary of the first category of patient information, the first scrollable text summary generated using the LLM based on the highlighted portion of the application.
